(19) United States
(12) Patent Application Publication
(10) Pub. No.: US 2014/0360495 A1
(43) Pub. Date: Dec. 11, 2014

(54) ARRANGEMENT IN THE FORM OF A TRACHEOSTOMA PROSTHESIS FOR EASY INSERTION AND EASY REMOVAL OF AN INNER CANNULA

(71) Applicant: Primed Halberstadt Medizintechnik GmbH, Halberstadt (DE)

(72) Inventors: Peter Traeger, Halberstadt (DE); Klaus Klimenta, Zilly (DE)

(73) Assignee: Primed Halberstadt Medizintechnik GmbH, Halberstadt (DE)

(21) Appl. No.: 14/363,176

(22) PCT Filed: Nov. 29, 2012
(86) PCT No.: PCT/DE2012/001182

§ 371 Date: Jun. 5, 2014

(30) Foreign Application Priority Data
Dec. 5, 2011 (DE) 10 2011 120 694.2

(51) Int. Cl. A61M 16/04

(52) U.S. Cl. CPC A61M 16/0427; A61M 16/0465; A61M 16/0497
USPC 128/200.26

(57) ABSTRACT

A tracheostoma prosthesis includes a tubular outer cannula with a proximal part and a distal part, a tubular inner cannula, wherein the inner cannula can be guided into the opening at the proximal part of the outer cannula and can he locked by locking or retaining means, and the outer edge of the proximal part of the outer cannula has two recesses lying opposite to each other, wherein the recesses are in the shape of an arc of a circle and are designed to receive a thumb and index finger of a hand for gripping the proximal end of the inner cannula at surfaces of the proximal end of the inner cannula exposed by the recesses, and the inner cannula has a bead-like edge at its proximal end.

### BACKGROUND OF THE INVENTION

The invention relates to an arrangement in the form of a tracheostoma prosthesis for the easy insertion and easy removal of an inner cannula.

Tracheostoma prostheses (also known as tracheostomy cannulas, tracheal cannulas, endotracheal tubes or tracheostomy tubes) for treating tracheostomized or laryngectomized patients with open throat (so called tracheostoma) have been known for decades.

From DE 195 14 433 A1 a tracheostomy cannula is known that is to be inserted in a tracheostoma and consists of a tubular outer cannula with a cannula shield and of a tubular inner cannula which can be guided into the outer cannula and locked with it at the proximal part.

This lock has the disadvantage that it can be possibly jammed and then it is difficult to release it so that the removal of the inner cannula from the outer cannula is considerably more complicated.

DE 38 19 237 A1 discloses an endotracheal tube comprising an outer tube and an inner tube and the inner tube is provided with a locking element for a snap-lock connection with the outer tube. The inner tube is also provided with an easily accessible operating handle which is connected with the locking element and to be used to pull out the inner tube from the connection with the outer tube.

This loop- or bracket-shaped operating handle has the disadvantage that it will tear off the inner tube, if the tensile forces are too high during the removal of the inner tube from the outer tube (e.g. if the inner tube is clamped in the outer tube or it is bonded by body secretions or the system is improperly handled) so that as a result of this tearing the inner tube can only be removed with very great effort from the outer tube.

The design patent 40208362-0001 and its description is related to a push-button connection for a treacheal cannula consisting of a six-teeth, round plastic part at the inner cannula and a round bead-like edge at the outer cannula. The inner cannula is released from the push-button connection with the outer cannula by levering the push-button connection. This is done by positioning the thumb and index finger laterally at the push-button connection and levering it out of the outer cannula. In order to fix the inner cannula in the outer cannula, the inner cannula is gently pressed on the push-button connection after inserting it in the outer cannula. It snaps into place with an audible click.

This push-button connection has the disadvantage that the inner cannula must be removed from the outer cannula by levering the push-button connection which is done by positioning the thumb and index finger laterally and then releasing said connection. When using the thumb and index finger for positioning them at the connection and subsequently levering, it, the nails of these lingers can be injured so that the handling of this push-button connection can be very awkward.

DE 202004 020 109 U1 discloses a treacheostoma tube with an adapter for lanryngectomy consisting of a tracheostoma tube equipped with an adapter at its distal end, and the free end of this adapter is provided with one recess or two oppositely arranged recesses at its edge. The adapter is used to add accessory parts (such as Trachi-Naze® (registered trademark of Kapitex Healthcare)) to a tracheostoma tube from the outside by coupling these accessory parts to the tracheostoma tube in a form-locked manner. The accessory parts can be very easily released from the adapter again by putting one finger of one hand into the recess or the thumb and index finger of one hand into the two recesses.

However, this technical solution has the disadvantage that it is not possible to insert an inner cannula in the outer cannula.

### SUMMARY OF THE INVENTION

The object of the invention is to make available an arrangement in the form of a tracheostoma prosthesis for the easy insertion and easy removal of an inner cannula, which arrangement avoids the disadvantages of prior art and in particular allows to pull out the inner cannula from a locked connection to the outer cannula with minimal effort by using the thumb and index finger, and this even when applying considerable tensile forces.

The essence of the invention is an arrangement in the form of a tracheostoma prosthesis that consists of an outer cannula and an inner cannula which can be inserted in this outer cannula, and the outer edge of the proximal part of the outer cannula is provided with two oppositely arranged molded recesses which are in the shape of an arc of a circle (so called grip-in recesses) and are designed to be receive a thumb and index finger of a hand for gripping the proximal end of the inner cannula at surfaces thereof exposed by the recesses, and the outer edge of the proximal part of the inner cannula has a bead-like shape.

### BRIEF DESCRIPTION OF THE DRAWINGS

In the following, the invention is explained in more detail by means of drawings. They show:

FIG. 1: a schematic overview drawing of an embodiment of an outer cannula of the inventive arrangement, and

FIG. 2: a lateral view of an embodiment of the inventive arrangement with an outer cannula and an inner cannula.

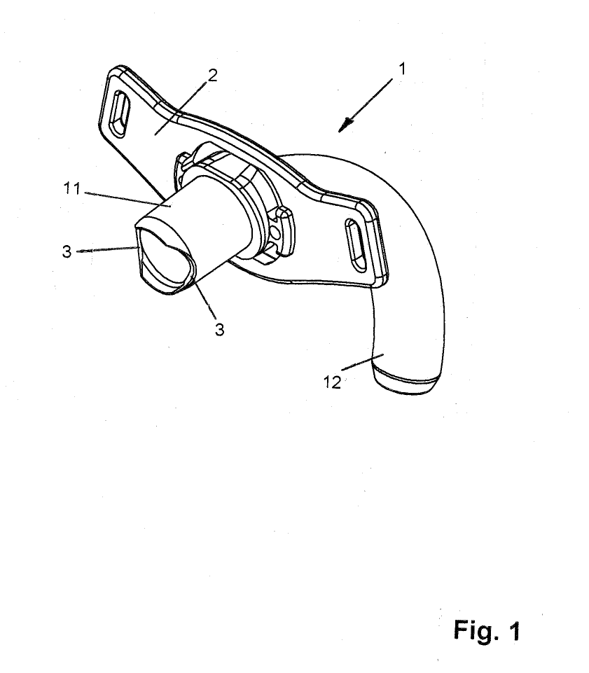

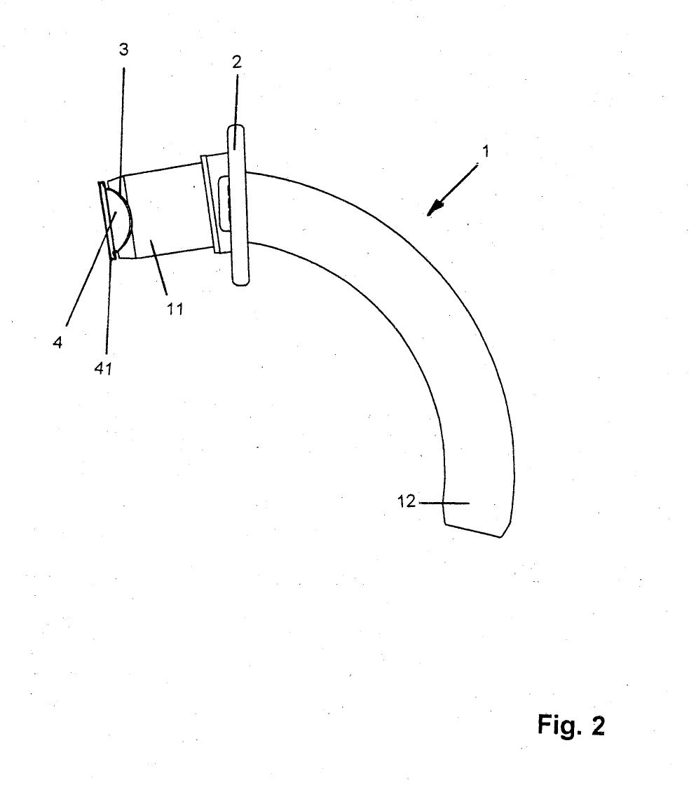

The outer cannula (1) shown in the FIGS. 1 and 2 has a tubular design according to the state of the art and is provided with a proximal part (11) and a distal part (12).

The cannula (4) represented in FIG. 2 has also a tubular design and is provided with a bead-like outer edge (41), which can also be referred to as an outer edge in a form of a bead, at its proximal end.

The inner cannula (4) can be guided in the opening at the proximal part (11) of the outer cannula (1) and can be locked in the proximal part (11) of the tubular outer cannula (1) by appropriate locking or retaining means (not shown in FIG. 2) according to the state of the art.

According to the state of the art, the tubular outer cannnula (1) can he provided with a cannula shield (2) at its proximal part (11).

An essential aspect of the invention is that the outer edge of the proximal part (11) of the outer cannula (1) is provided with two circular-arc shaped, molded recesses (3) positioned opposite to each other, so called grip-in recesses, designed to be gripped by thumb and index finger, and that the inner cannula (4) has an outer edge (41) with a bead-like design at its proximal end.

These recesses (3) and the bead-like outer edge (41) have the advantage that an inner cannula (4) locked in the outer cannula (1) according to the state of the art can be easily and without any difficulty—even with strong tensile forces removed from the outer cannula (1) by the direct gripping of thumb and index finger in the bead-like edge (41) of the proximal end of the inner cannula (4) with said proximal end being exposed in the area of the recesses (3). When doing this, neither the nails of thumb and index finger are damaged nor components of the inner cannula, e.g. the operating handle, are destructively removed.

Thus, the thumb and index finger grip in a very ergonomic manner in the area of the recesses (3), i.e. the grip-in molds, at the edge (41) of the inner cannula (4) so that the tensile forces applied by the thumb and index finger can be optimally transmitted to the inner cannula (4) to remove it from the outer cannula (1) by pulling it out.

The outer cannula (1) and the inner cannula (4) are made of plastic material, such as PVC, silicone or thermoplastic polyurethane, or of metal, e.g. sterling silver.

According to the state of the art, the outer cannula (1) and the inner cannula (4) can be designed as a standard arc from 80° to 110°.

The individual features or any combination of them described in this invention and the following claims can be decisive for the invention.

1. A tracheostoma prosthesis comprising a tubular outer cannula having a proximal part and a distal part, and a tubular inner cannula which can be guided into an opening at the proximal part of the outer cannula and can be locked retained, wherein an outer edge of the proximal part of the outer cannula is provided with two recesses arranged opposite to each other, the recesses having the shape of an arc of a circle and being configured to receive a thumb and index finger of a hand for gripping a proximal end of the inner cannula at surfaces thereof exposed by the recesses, and the inner cannula has an edge in a form of a bead.

2. The tracheostoma prosthesis according to claim 1, wherein the outer cannula is provided with a cannula shield at its proximal part.

3. The tracheostoma prosthesis according to claim 1, wherein the outer cannula and the inner cannula are made of plastic material.

4. The tracheostoma prosthesis according to claim 1, wherein the outer cannula and the inner cannula are each configured as a 80° to 110° arc.

5. The tracheostoma prosthesis according to claim 3, wherein the plastic material is PVC, silicone or thermoplastic polyurethane and the metal is sterling silver.
